(19) United States
(12) Patent Application Publication
(10) Pub. No.: US 2009/0004208 A1
(43) Pub. Date: Jan. 1, 2009

(54) Method for designing hypoallergenic molecules for use in allergy desensitization with lessened chance of anaphylaxis, or as vaccines against allergic reactions

(76) Inventor: Eduardo A. Padlan, Kensington, MD (US)

Correspondence Address: Eduardo A. Padlan, 4006 Simms Drive, Kensington, MD, 20895, US

(21) Appl. No.: 11/823,330

(22) Filed: Jun. 28, 2007

(51) Int. Cl. A61K 39/00; C07K 2/00; C07H 21/04; A61K 38/02; A61K 31/7052

(52) U.S. Cl. 424/184.1; 530/300; 536/23.1; 514/2; 514/44

(57) ABSTRACT

A unique method is disclosed for identifying and replacing surface amino acid residues of a protein allergen that reduces the antigenicity of its dominant IgE epitopes. The method is useful in the design of hypoallergenic molecules for use in allergy desensitization with lessened chance of anaphylaxis, or as vaccines against allergic reactions.

### REFERENCES CITED

Benjamin, D. C. et al., The antigenic structure of proteins: a reappraisal, 1984, Annu. Rev. Immunol., 2, pp. 67-101.
Berman, H. M. et al., The Protein Data Bank, 2000, Nuc. Acids Res., 28, pp. 235-242.
Bohle, B., et al., Characterization of the human T cell response to antigen 5 from Vespula vulgaris (Ves v 5), 2005, Clin. Exp. Allergy, 35, pp. 367-373.
Czerwinski E. W. et al., Crystal structure of Jun a 1, the major cedar pollen allergen from Juniperus ashei, reveals a parallel beta-helical core, 2005, J. Biol. Chem., 280, pp. 3740-3746.
David, M. P. et al., A study of the structural correlates of affinity maturation: Antibody affinity as a function of chemical interactions, structural plasticity and stability, 2007, Mol. Immunol., 44, pp. 1342-1351.
Davies, D. R. et al., Antibody-antigen complexes, 1988, J. Biol. Chem., 263, pp. 10541-10544.
De Genst, E. et al., Kinetic and affinity predictions of a protein-protein interaction using multivariate experimental design, 2002, J. Biol. Chem., 277, pp. 29897-29907.
de Halleux, S. et al., Three-dimensional structure and IgE-binding properties of mature fully active Der p 1, a clinically relevant major allergen, 2006, J. Allergy Clin. Immunol., 117, pp. 571-576.
Ferreira, F. et al., Dissection of immunoglobulin E and T lymphocyte reactivity of isoforms of the major birch pollen allergen Bet v 1: potential use of hypoallergenic isoforms for immunotherapy, 1996, J. Exp. Med., 183, pp. 599-609.
Ferreira, F. et al., Modulation of IgE reactivity of allergens by site-directed mutagenesis: potential use of hypoallergenic variants for immunotherapy, 1998, FASEB J., 12, pp. 231-242.
Grantham, R., Amino acid difference formula to help explain protein evolution, 1974, Science, 185, pp. 862-864.
Henriksen, A. et al., Major venom allergen of yellow jackets, Ves v 5: Structural characterization of a pathogenesis-related protein superfamily, 2001, PROTEINS: Struct., Funct., Genet., 45, pp. 438-448.
Kabsch, W. et al., Dictionary of protein secondary structure: pattern recognition of hydrogen-bonded and geometrical features, 1983, Biopolymers, 22, pp. 2577-2637.
Novotny, J., Protein antigenicity: a thermodynamic approach, 1991, Mol. Immunol., 28, pp. 201-227.
Pace, C. N. et al., A helix propensity scale based on experimental studies of peptides and proteins, 1998, Biophys. J., 75, pp. 422-427.
Padlan, E. A., Quantitation of the immunogenic potential of protein antigens, 1985, Mol. Immunol., 22, pp. 1243-1254.
Padlan, E. A. On the Nature of Antibody Combining Sites: Unusual Structural Features That May Confer on These Sites an Enhanced Capacity for Binding Ligands, 1990, PROTEINS: Struct. Funct. Genet., 7, pp. 112-124.
Padlan, E. A., Anatomy of the Antibody Molecule, 1994, Mol. Immunol., 31, pp. 169-217.
Padlan, E. A. X-ray Crystallography of Antibodies, 1996, Adv. Prot. Chem., 49, pp. 57-133.
Rammensee, H. et al., SYFPEITHI: database for MHC ligands and peptide motifs, 1999, Immunogenetics, 50, pp. 213-219.
Sandberg, M. et al., New chemical descriptors relevant for the design of biologically active peptides. A multivariate characterization of 87 amino acids, 1998, J. Med. Chem., 41, pp. 2481-2491.
Schramm, G. et al., “Allergen Engineering”: Variants of the Timothy Grass Pollen Allergen Phl p 5b with Reduced IgE-Binding Capacity but Conserved T Cell Reactivity, 1999, J. Immunol. 162, pp. 2406-2414.
Sneath, P. H., Relations between chemical structure and biological activity in peptides, 1966, J. Theor. Biol., 12, pp. 157-195.
Street, A. G. et al., Intrinsic beta-sheet propensities result from van der Waals interactions between side chains and the local backbone, 1999, Proc. Natl. Acad. Sci. U.S.A., 96, pp. 9074-9076.
Vrtala, S. et al., Conversion of the Major Birch Pollen Allergen, Bet v 1, into Two Nonanaphylactic T Cell Epitope-containing Fragments Candidates for a Novel Form of Specific Immunotherapy, 1997, J. Clin. Invest., 99, pp. 1673-1681.

### BRIEF DESCRIPTION OF THE DRAWINGS

Table 1 shows the structural characteristics of the different amino acids and the amino-acid replacements designed to reduce the antigenicity of protein epitopes.

FIG. 1 shows the plots of antigenicity vs residue position for the European house dust mite allergen, Der p 1, before (top) and after two rounds of de-Antigenization (bottom).

FIG. 2 shows the plots of antigenicity vs residue position for the major cedar pollen allergen, Jun a 1, before (top) and after four rounds of de-Antigenization (bottom).

FIG. 3 shows the plots of antigenicity vs residue position for the major yellow jackets venom allergen, Ves v 5, before (top) and after four rounds of de-Antigenization (bottom).

### FIELD OF THE INVENTION

This invention relates to the design of hypoallergenic molecules that could be used in the desensitization of allergic individuals with lessened chance of anaphylaxis. The hypoallergenic molecules may also be used as vaccines against allergy.

### BACKGROUND OF THE INVENTION

When we are exposed to a foreign substance (an antigen), our immune system reacts by producing molecules and cells that are specific for the substance. Antibodies are molecules produced by our immune system and these bind the antigen, neutralizing or immobilizing it, and, thereby, rendering it more susceptible to elimination by normal processes. Various types of antibodies are produced by the immune system and the various antibody types have different structures, functions, and distribution in our body. For example, the major type of antibody that we produce is IgG (Immunoglobulin G). A type of antibody that is produced in much smaller amounts is IgE; IgE is the antibody type that is responsible for allergy. It is not known why some antigens elicit an IgE response and not an IgG response. It is also not known why some individuals, when exposed to a particular antigen, develop an allergic reaction to it, while others don't. Antigens that elicit an IgE response are called allergens.

A number of cell types have receptors for IgE on their surface. For example, mast cells, that lie under our skin and in the lining of our blood vessels, and basophils, that circulate in our blood, bind IgE through high affinity receptors. When allergen binds to IgE on mast cells, or basophils, the cells release histamine and other vasoactive compounds from pre-formed granules in their cytoplasm. The release of those molecules results in the usual allergic symptoms: sneezing, coughing, rashes, local edema, etc. Severe allergic reactions, like edema that closes the breathing passages, or systemic anaphylaxis, could result in death.

An attempt to rid an individual of allergy to a particular allergen is made by exposing the individual to ever increasing amounts of allergen over time—a process called desensitization. The objective of desensitization is to elicit an IgG response that would compete with IgE for the allergen. Not surprisingly, there is danger that desensitization could cause a severe allergic reaction.

Various attempts have been made to produce allergens with reduced allergenicity (the antigenicity of an allergen; here, antigenicity, the ability to elicit an antibody response, and allergenicity, the ability to elicit an IgE response, are used interchangeably) (see, for example, Ferreira et al., 1996; Vrtala et al., 1997; Ferreira et al., 1998; Schramm et al., 1999). Such hypoallergenic molecules would permit safer desensitization. If the regions in an allergen, to which the IgE molecules bind (the dominant IgE epitopes) are known, the residues in those regions could be replaced by amino acids that would cause less binding to IgE.

The method described here is a purely computational procedure designed to locate the putative dominant IgE epitopes (putative because it is impossible to identify and delineate all the dominant IgE epitopes of any allergen) and to identify the residues which contribute to the antigenicity of those epitopes. The method, called “de-Antigenization”, also describes a procedure to decrease the antigenicity of the dominant IgE epitopes by the judicious replacement of the contributing residues with amino acids that by virtue of their physicochemical properties are expected to contribute less to antigenicity.

### SUMMARY OF THE INVENTION

The de-Antigenization of the putative dominant IgE epitopes is achieved through the following steps:

(Step 1) Identify a protein molecule that has been identified as a major allergen.

(Step 2) Calculate the antigenicity of the various regions of the allergen, using three-dimensional structural information about the molecule and the known physicochemical properties of the amino-acid residues. Locate the regions with high antigenicities, i.e. the putative dominant IgE epitopes.

(Step 3) Identify the amino-acid residues comprising the putative dominant IgE epitopes, in particular those residues which, by virtue of their physicochemical properties and their accessibility, can contribute significantly to tight binding by IgE. Replace those residues with amino acids that would be expected to contribute less to the binding by IgE, while ensuring that the replacements will not significantly alter the structure of the allergen. At least one T-cell epitope should be preserved.

(Step 4) Using the new structure (the structure with the replacements), repeat Steps 2 and 3 as needed until the putative dominant IgE epitopes have significantly lower antigenicities.

(Step 5) The amino acid sequences, which result in significantly lower antigenicities for the putative dominant IgE epitopes, and polynucleotides derived from those sequences, provide the basis for hypoallergenic molecules that could be used in the desensitization of allergic individuals with lessened chance of anaphylaxis, or as vaccines against the allergy.

### DETAILED DESCRIPTION OF THE INVENTION

Information about the three-dimensional structure of a particular allergen is often available from the Protein Data Bank (Berman et al., 2000) (http://www.rcsb.org/pdb). In the absence of experimentally-determined three-dimensional information, a model of the allergen could be built based on structural information from closely related molecules. Various techniques are available for modeling purposes and those techniques are known to those skilled in the art.

On the basis of the three-dimensional structure of the allergen, the solvent accessibilities of the individual amino acid residues are computed using standard methods (see, for example, Padlan, 1990; Padlan 1994). Solvent accessibilities could also be obtained using the program DSSP (Kabsch et. al., 1983) (implemented in http://bioweb.pasteur.fr/seqanal/interfaces/dssp-simple.html). The solvent accessibilities are used as weighting factors in the calculation of the antigenicities. The use of solvent accessibilities as weighting factors de-emphasizes the contribution of residues that are not too accessible and that probably do not contribute much to the interaction with IgE.

A method had been proposed earlier for quantifying the antigenicity of a given region in a protein molecule using the physicochemical attributes of the amino acid residues in the region (Padlan, 1985). That method is particularly suitable for locating the putative dominant IgE epitopes and is followed here. Structural parameters describing the physicochemical attributes of the various amino acids have been computed by various authors (for example, Sneath, 1966; Grantham, 1974; Sandberg et al., 1998) and those can be used in the calculation of antigenicities. The antigenicity of a region in the molecule is computed by taking the sum of the structural parameters, weighted or unweighted, corresponding to all the residues within that region. Structural parameters have been shown to provide a good measure of the ability of a given region to participate in antibody-antigen and other protein-protein interactions (see, for example, Padlan, 1990; Novotny, 1991; be Genst et al., 2002; David et al., 2007). Thus, antigenicity computed in this manner is directly correlated with the ability of a particular region to engage in tight binding to IgE. The regions displaying highest antigenicities are identified as the putative dominant IgE epitopes.

The de-Antigenization of the putative dominant IgE epitopes is achieved by the judicious replacement of the residues in those epitopes with amino acids that would contribute less to the total antigenicity values, while preserving the structure of the molecule. By taking into account the physicochemical properties of the amino acids and their propensity to participate in a particular secondary structure (presented in Table 1), replacement rules could be proposed. The replacement rules used in the examples below are included in Table 1. Other replacement rules could be proposed and used provided that they result in reduced antigenicity while preserving structure.

The concept can be implemented by those skilled in the art using the following, or similar, algorithm:

(A.1.0)—Generate a set of amino-acid replacement rules based on structural criteria, e.g., the replacement rules in Table 1. The recommended structural criteria are (1) the replacing amino acid should contribute less to the binding interaction with an antibody and (2) the replacement should not result in a significant change in the structure of the molecule.

(A.2.0)—Identify a protein molecule that is a major allergen in a particular allergy. Locate on the sequence the known T-cell epitopes of the molecule; if T-cell epitopes had not been experimentally determined, obtain possible T-cell epitopes using predictors, e.g. SYFPEITHI (Rammensee et al., 1999) (http://www.syfpeithi.de). If an experimentally-determined three-dimensional structure is available for the allergen, proceed to (A.3.0);

(A.2.1)—If a model structure for the allergen is available, proceed to (A.3.0);

(A.2.2)—Identify a homologous molecule for which an experimentally-determined three-dimensional structure or a model structure is available; if there is none, STOP

(A.2.3)—Generate a model for the allergen from its amino acid sequence.

(A.3.0)—Generate atomic coordinates for the biological, i.e. natural, aggregation state of the molecule (dimer, trimer, etc.) using appropriate symmetry operations. For experimentally-determined structures, atomic coordinates for the biological aggregation state may already be available from the Protein Data Bank. All subsequent computations will be on the biological aggregation state of the molecule.

(A.4.0)—Choose and isolate the positions at which the antigenicities will be computed, e.g., the alpha-carbon positions.

(A.5.0)—Compute the solvent accessibilities of the individual amino acid residues by using standard procedures (as described in Padlan, 1990 and references cited therein), or by using program DSSP (Kabsch et al., 1983) (implemented, for example, in http://bioweb.pasteur.fr/seqanal/interfaces/dssp-simple.html).

(A.6.0)—Choose a set of structural parameters (physicochemical attributes) for use in the computation of the antigenicities. The structural parameters compiled by Sandberg et al. (1998), or by Grantham (1974), are particularly suitable for the computation of antigenicities.

(A.7.0)—Compute the antigenicities at the positions chosen in (A.4.0). A measure of antigenicity ascribed to a given position would be the total contribution of the amino acids within a defined region around that position. The contribution of each amino acid may be the sum, appropriately weighted or unweighted, of the structural parameters chosen in (A.6.0). The solvent accessibility of the amino acid, computed in (A.5.0), is recommended as an appropriate overall weight for the contribution of that amino acid to the antigenicity.

(A.8.0)—Identify the possible location of the putative dominant IgE epitopes. The positions with antigenicity values significantly higher than the rest are most probably part of the putative dominant IgE epitopes. A basis for the identification of the putative dominant IgE epitopes, could be the root-mean-square (r.m.s.) deviation from the mean of the antigenicity values of all epitopes.

(A.9.0)—Replace the residues comprising the putative dominant IgE epitopes according to the replacement rules generated in (A.1.0). The residues would be the ones located within a certain radius of the epitope centers chosen in (A.4.0). A suitable value for the radius could be determined by examining known antibody-antigen complexes (see, for example, Padlan, 1996). It is recommended that the residues to be replaced be chosen on the basis of their solvent accessibility and their relative contribution to the overall antigenicity of the epitope. Preserve those residues which are probably critical to the structure (secondary, tertiary, quaternary) of the antigen, including residues whose posttranslational modification, e.g. glycosylation, is probably required for preservation of structure. Preserve at least one of the T-cell epitopes located in (A.2.0), as well as segments for which high antigenicity values might elicit useful antibody responses, e.g. inhibition of particular reactions. The suggested replacement should not be made if it will result in a peptide segment (of sufficient length to be presented by T cells) that is identical to a segment present in a human protein; this is to obviate autoimmune reactions.

(A.10.0)—Repeat (A.2.3) to (A.9.0) until it is deemed that the decrease in antigenicity of the putative dominant IgE epitopes is sufficient, or until no further amino-acid replacements are warranted.

(A.11.0)—The amino acid sequences resulting from (A.10.0), or the polynucleotides derived from those sequences, provide the basis for hypoallergenic molecules that could be used in the desensitization of allergic individuals, with lessened chance of anaphylaxis, or as vaccines against the allergy.

The present invention will now be described with reference to the following specific, non-limiting examples.

### EXAMPLE 1

Design of possible hypoallergenic molecules for use in the desensitization with lessened chance of anaphylaxis, or as vaccines, against Der p 1, the major allergen of the European house dust mite, Dermatophagoides pteronyssinus:

### Structural and Sequence Data:

Three-dimensional structural information for the mature form of Der p 1 has been provided by X-ray crystallography (de Halleux et al., 2006) (Protein Data Bank entry 2AS8). The sequence of the mature form of Der p 1, for which an X-ray structure is available, is presented as SEQ ID NO: 1. Hereinafter, the fragment represented by that structure will be referred to simply as 2AS8. Using SYFPEITHI, three putative T-cell epitopes were predicted: residues 22-36, 34-48, and 37-51. During de-Antigenization, residues 22-51 were preserved.

### Solvent Accessibilities:

The solvent accessibilities of the individual residues of 2AS8 were obtained using the program DSSP (Kabsch et al., 1983) (http://bioweb.pasteur.fr/seqanal/interfaces/dssp-simple.html). Fractional accessibility for each amino acid was estimated by dividing the accessibility obtained from DSSP by the total surface area of the amino acid (obtained from http://prowl.rockefeller.edu/aainfo/volume.htm).

### Calculation of Antigenicities and Identification of the Dominant IgE Epitopes:

The structural parameters provided by Sandberg et al. (1998) (reproduced in Table 1) were used in the calculation of antigenicities. The antigenicity of a region centered at each alpha-carbon position was computed by taking the sum of the zz1, zz2 and zz3 structural parameters of Sandberg et al. (1988) corresponding to all the residues within 14 Angstroms of the alpha-carbon. In this example, the radius of 14 Angstroms was chosen on the basis of the results of calculations on the known epitopes of the allergen, hen egg white lysozyme (Padlan, 1996). The solvent accessibilities obtained above for 2AS8 were used as weighting factors in the calculation of the antigenicities.

### De-Antigenization of the Dominant IgE Epitopes:

Only those epitopes whose antigenicity values are greater than 2 r.m.s. deviations above the mean were considered. De-Antigenization was achieved after two rounds of antigenicity calculation followed by amino-acid replacements. No further replacements were suggested after the two rounds. The replacement rules proposed in Table 1 were applied. Only those residues, whose contribution to the antigenicity of the putative dominant IgE epitope is at least 3% of the total and whose fractional solvent accessibility is at least 40%, were replaced.

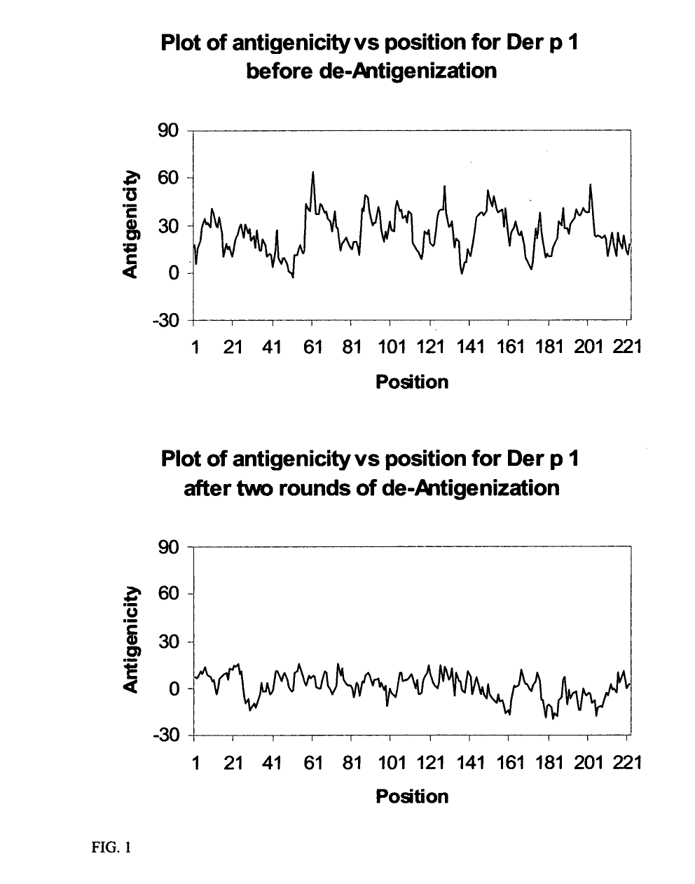

Prior to de-Antigenization, the average antigenicity of the molecule represented by SEQ ID NO: 1 was 25.5 (r.m.s. deviation=12.6) (arbitrary units). A total of 27 amino acid replacements were made, yielding SEQ ID NO: 2. This resulted in an average antigenicity value of 2.3 (r.m.s. deviation=8.4); 2 more changes were suggested, yielding SEQ ID NO: 3. This resulted in an average antigenicity value of 1.6 (r.m.s. deviation=7.9); no more changes were suggested. The plots of antigenicities computed for 2AS8, before and after two rounds of de-Antigenization, are presented in FIG. 1.

Possible Hypoallergenic Molecule for Use in the Desensitization to Der p 1, with Lessened Chance of Anaphylaxis, or as Vaccine Against Allergy to the European House Dust Mite:

Since every round of de-Antigenization resulted in a significant decrease in the antigenicity of the dominant IgE epitopes, any of the derivative amino-acid sequences (SEQ ID NO: 2 or 3), or a polynucleotide derived from it, could be the basis of a possible hypoallergenic molecule useful in the desensitization of individuals allergic to Der p 1 with lessened chance of anaphylaxis, or as possible vaccine against European house dust mite allergy. The best candidate is probably the one represented by the sequence after the two rounds of de-Antigenization (SEQ ID NO: 3).

### EXAMPLE 2

Design of possible hypoallergenic molecules for use in the desensitization with lessened chance of anaphylaxis, or as vaccines, against Jun a 1, the major pollen allergen from the cedar, Juniperus ashei:

### Structural Data:

A crystallographically-determined structure of Jun a 1 (Czerwinski et al., 2005) is available from the Protein Data Bank (Entry 1PXZ), hereinafter referred to simply as 1PXZ. The sequence of the mature form of Jun a 1, for which an X-ray structure is available, is presented as SEQ ID NO: 4. Several peptides were predicted by SYFPEITHI as possible T-cell epitopes; two of these (residues 131-145 and 142-156) were chosen to be preserved during de-Antigenization.

### Solvent Accessibilities:

Solvent accessibilities for 1PXZ were computed as in EXAMPLE 1. The surface areas accessible to solvent were computed using DSSP and the fractional accessibility of each residue was estimated by dividing the solvent accessible area of the residue by the surface area of the particular amino acid.

### Calculation of Antigenicities and Identification of the Dominant IgE Epitopes:

The antigenicity of regions around the alpha-carbon positions of 1PXZ were computed as in EXAMPLE 1. The zz1, zz2 and zz3 structural parameters of Sandberg et al. (1998) were used. A radius of 14 Angstroms was used to define the region around each alpha-carbon position. The initial average antigenicity value was 22.5 (arbitrary units) with a root-mean-square (r.m.s.) deviation from the mean of 12.2. The regions with antigenicity values greater than two r.m.s. deviations above the mean were identified as the putative dominant IgE epitopes.

### De-Antigenization of the Dominant IgE Epitopes:

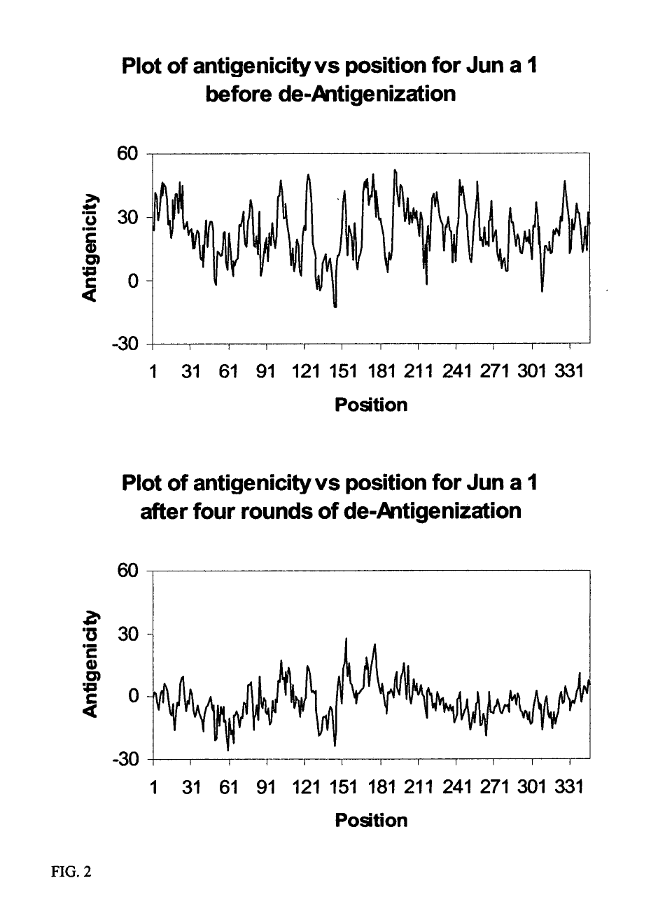

The residues in the putative dominant IgE epitopes, which each contribute at least 3% of the total antigenicity of the epitope and whose fractional accessibilities are greater than 40%, were replaced according to the rules proposed in Table 1. Seventeen residues were replaced, yielding SEQ ID NO: 5. The antigenicities were recalculated and this resulted in an average antigenicity of 12.1 (r.m.s. deviation=11.6). Fourteen more residues were replaced, yielding SEQ ID NO: 6. This resulted in an average antigenicity of 4.2 (r.m.s. deviation=7.8). Eleven more residues were replaced, yielding SEQ ID NO: 7. A third round of de-Antigenization resulted in an average antigenicity of −0.3 (r.m.s. deviation=8.3). After replacing six more residues, yielding SEQ ID NO: 8, a fourth round of de-Antigenization resulted in an average antigenicity of −2.1 (r.m.s. deviation=8.3). No additional residues were found to need replacement after this fourth round of de-Antigenization. The antigenicities before and after the four rounds of de-Antigenization of 1PXZ are plotted in FIG. 2.

Possible Hypoallergenic Molecules for Use in the Desensitization, with Lessened Chance of Anaphylaxis, or as Vaccines Against Jun a 1, the Major Pollen Allergen from the Cedar, Juniperus ashei:

Since every round of de-Antigenization resulted in a significant decrease in the antigenicity of the dominant IgE epitopes, any of the derivative amino-acid sequences (SEQ ID NO: 5 through 8), or a polynucleotide derived from it, could be the basis of a possible hypoallergenic molecule useful in the desensitization of individuals allergic to Jun a 1 with lessened chance of anaphylaxis, or as possible vaccine against pollen from Juniperus ashei. The best candidate is probably the one represented by the sequence after the four rounds of de-Antigenization (SEQ ID NO: 8).

### EXAMPLE 3

Design of possible hypoallergenic molecules for use in the desensitization with lessened chance of anaphylaxis, or as vaccines, against Ves v 5, the major venom allergen from yellow jackets, Vespula vulgaris:

### Structural Data:

A crystallographically-determined structure of Ves v 5 (Henriksen et al., 2001) is available from the Protein Data Bank (Entry 1QNX), hereinafter referred to simply as 1QNX. The sequence of the mature form of Ves v 5, for which an X-ray structure is available, is presented as SEQ ID NO: 9. Several peptides have been shown to be T-cell epitopes (Bohle et al., 2005); two of those (residues 78-87 and 181-192) were chosen to be preserved during de-Antigenization.

### Solvent Accessibilities:

Solvent accessibilities for 1QNX were computed as in EXAMPLE 1. The surface areas accessible to solvent were computed using DSSP and the fractional accessibility of each residue was estimated by dividing the solvent accessible area of the residue by the surface area of the particular amino acid.

### Calculation of Antigenicities and Identification of the Dominant IgE Epitopes:

The antigenicity of regions around the alpha-carbon positions of 1QNX were computed as in EXAMPLE 1. The zz1, zz2 and zz3 structural parameters of Sandberg et al. (1998) were used. A radius of 14 Angstroms was used to define the region around each alpha-carbon position. The initial average antigenicity value was 12.1 (arbitrary units) with a root-mean-square (r.m.s.) deviation from the mean of 11.2. The regions with antigenicity values greater than two r.m.s. deviations above the mean were identified as the putative dominant IgE epitopes.

### De-Antigenization of the Dominant IgE Epitopes:

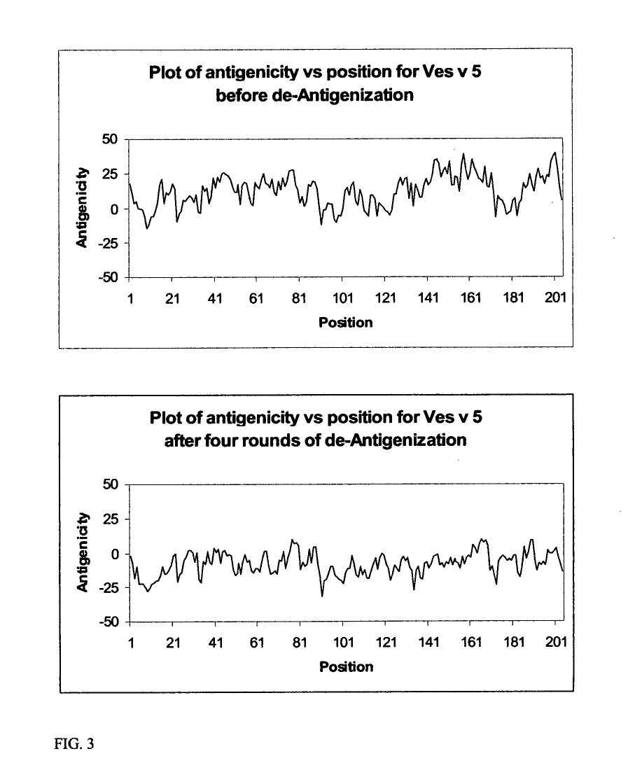

The residues in the putative dominant IgE epitopes, which each contribute at least 3% of the total antigenicity of the epitope and whose fractional accessibilities are greater than 40%, were replaced according to the rules proposed in Table 1. Twelve residues were replaced, yielding SEQ ID NO: 10. The antigenicities were recalculated and this resulted in an average antigenicity of 2.9 (r.m.s. deviation=10.5). Seven more residues were replaced, yielding SEQ ID NO: 11. This resulted in an average antigenicity of −2.7 (r.m.s. deviation=10.6). Eleven more residues were replaced, yielding SEQ ID NO: 12. A third round of de-Antigenization resulted in an average antigenicity of −6.1 (r.m.s. deviation=9.3). After replacing two more residues, yielding SEQ ID NO: 13, a fourth round of de-Antigenization resulted in an average antigenicity of −8.1 (r.m.s. deviation=8.2). No additional residues were found to need replacement after this fourth round of de-Antigenization. The antigenicities before and after the four rounds of de-Antigenization of 1QNX are plotted in FIG. 3.

Possible Hypoallergenic Molecules for Use in the Desensitization, with Lessened Chance of Anaphylaxis, or as Vaccines Against Ves v 5, the Major Venom Allergen from the Cedar, Vespula vulgaris:

Since every round of de-Antigenization resulted in a significant decrease in the antigenicity of the dominant IgE epitopes, any of the derivative amino-acid sequences (SEQ ID NO: 9 through 13), or a polynucleotide derived from it, could be the basis of a possible hypoallergenic molecule useful in the desensitization of individuals allergic to Ves v 5 with lessened chance of anaphylaxis, or as possible vaccine against pollen from Vespula vulgaris. The best candidate is probably the one represented by the sequence after the four rounds of de-Antigenization (SEQ ID NO: 13).

TABLE 1
The amino acid parameters used in the calculation of antigenicities and the replacement suggestions

| Amino | | | | | | | Helix | Sheet | Coil | Turn | If in Helix | Sheet | Coil | Turn |
|---|---|---|---|---|---|---|---|---|---|---|---|---|---|---|
| acid | zz1 | zz2 | zz3 | zz4 | zz5 | SDGly | Propensities | | | | Change to: | | | |
| Ala | 0.24 | −2.32 | 0.60 | −0.14 | 1.30 | 60.0 | 0.00 | 0.47 | −0.26154 | 0.83 | — | — | — | — |
| Arg | 3.52 | 2.50 | −3.50 | 1.99 | −0.17 | 125.0 | 0.21 | 0.35 | −0.17659 | 0.82 | Ala | Thr | Ala | Ala |
| Asn | 3.05 | 1.62 | 1.04 | −1.15 | 1.61 | 80.0 | 0.65 | 0.40 | 0.22989 | 1.44 | Ala | Thr | Ser | Gly |
| Asp | 3.98 | 0.93 | 1.93 | −2.46 | 0.75 | 94.0 | 0.69 | 0.72 | 0.22763 | 1.41 | Ala | Thr | Ser | Gly |
| Cys | 0.84 | −1.67 | 3.71 | 0.18 | −2.65 | 159.0 | 0.68 | 0.25 | −0.015152 | 1.08 | — | — | — | — |
| Gln | 1.75 | 0.50 | −1.44 | −1.34 | 0.66 | 87.0 | 0.39 | 0.34 | −0.187677 | 0.94 | Ala | Thr | Ala | Thr |
| Glu | 3.11 | 0.26 | −0.11 | −3.04 | −0.25 | 98.0 | 0.40 | 0.35 | −0.20469 | 1.01 | Ala | Thr | Ala | Thr |
| Gly | 2.05 | −4.06 | 0.36 | −0.82 | −0.38 | 0.0 | 1.00 | — | 0.43323 | 1.48 | — | — | — | — |
| His | 2.47 | 1.95 | 0.26 | 3.90 | 0.09 | 98.0 | 0.56 | 0.37 | −0.0012174 | 1.07 | Ala | Thr | Thr | Thr |
| Ile | −3.89 | −1.73 | −1.71 | −0.84 | 0.26 | 135.0 | 0.41 | 0.10 | −0.42224 | 0.59 | — | — | — | — |
| Leu | −4.28 | −1.30 | −1.49 | −0.72 | 0.84 | 138.0 | 0.21 | 0.32 | −0.33793 | 0.66 | — | — | — | — |
| Lys | 2.29 | 0.89 | −2.49 | 1.49 | 0.31 | 127.0 | 0.26 | 0.34 | −0.100092 | 1.01 | Ala | Thr | Thr | Thr |
| Met | −2.85 | −0.22 | 0.47 | 1.94 | −0.98 | 127.0 | 0.24 | 0.26 | −0.22590 | 0.57 | — | — | — | — |
| Phe | −4.22 | 1.94 | 1.06 | 0.54 | −0.62 | 153.0 | 0.54 | 0.13 | −0.22557 | 0.89 | Ala | Thr | Ala | Ala |
| Pro | −1.66 | 0.27 | 1.84 | 0.70 | 2.00 | 42.0 | 3.01 | — | 0.55232 | 1.38 | — | — | — | — |
| Ser | 2.39 | −1.07 | 1.15 | −1.39 | 0.67 | 56.0 | 0.50 | 0.30 | 0.14288 | 1.15 | — | — | — | — |
| Thr | 0.75 | −2.18 | −1.12 | −1.46 | −0.40 | 59.0 | 0.66 | 0.06 | 0.0088780 | 1.00 | — | — | — | — |
| Trp | −4.36 | 3.94 | 0.59 | 3.44 | −1.59 | 184.0 | 0.49 | 0.24 | −0.243375 | 0.70 | Ala | Thr | Ala | Val |
| Tyr | −2.54 | 2.44 | 0.43 | 0.04 | −1.47 | 147.0 | 0.53 | 0.11 | −0.20751 | 0.92 | Ala | Thr | Ala | Thr |
| Val | −2.59 | −2.64 | −1.54 | −0.85 | −0.02 | 109.0 | 0.61 | 0.13 | −0.38618 | 0.70 | — | — | — | — |

Footnote to Table 1:
The zz values are from Sandberg et al. (1998).
The SDGly values are from Grantham (1974) and represent the structural dissimilarities of the various amino acids relative to glycine.
The helix propensities are from Pace et al. (1998).
The beta sheet propensities are from Street et al. (1999).
The coil propensities are from Linding et al. (2003).
The turn propensities are from Hutchinson et al. (1994). A dash in the replacement suggestions signifies that no change is recommended.

What are claimed are:

1. A method for reducing the antigenicity of putative dominant IgE epitopes in a protein allergen, the method comprising:
a) identifying the putative dominant IgE epitopes of the allergen and the amino acid residues which constitute those epitopes; and
b) replacing the residues, which contribute the most to the antigenicity of the putative dominant IgE epitopes, with amino acids whose physicochemical properties will effectively reduce the antigenicity of those epitopes while preserving structure.

2. A polypeptide designed using claim 1.

3. A polynucleotide derived from a polypeptide of claim 2.

4. A pharmaceutical composition comprising the polypeptide of claim 2 and a pharmaceutically acceptable carrier.

5. A pharmaceutical composition comprising the polynucleotide of claim 3 and a pharmaceutically acceptable carrier.

6. A pharmaceutical composition of claim 4 that is used in the desensitization of an individual against allergen.

7. A pharmaceutical composition of claim 4 that is used as a vaccine against allergy.

8. A pharmaceutical composition of claim 5 that is used in the desensitization of an individual against allergen.

9. A pharmaceutical composition of claim 5 that is used as a vaccine against allergy.

10. A method for reducing the antigenicity of IgE epitopes in a protein allergen that is based on the method described in claim 1.

11. A polypeptide designed using a method described in claim 10.

12. A polynucleotide derived from a polypeptide of claim 11.

13. A pharmaceutical composition comprising the polypeptide of claim 11 and a pharmaceutically acceptable carrier.

14. A pharmaceutical composition comprising the polynucleotide of claim 12 and a pharmaceutically acceptable carrier.

15. A pharmaceutical composition of claim 13 that is used in the desensitization of an individual against allergen.

16. A pharmaceutical composition of claim 13 that is used as a vaccine against allergy.

17. A pharmaceutical composition of claim 14 that is used in the desensitization of an individual against allergen.

18. A pharmaceutical composition of claim 14 that is used as a vaccine against allergy.


SEQUENCE LISTING (13 sequences)

SEQ ID NO: 1 - protein, 222 aa, Dermatophagoides pteronyssinus
  2AS8, mature form of Der p 1, the major allergen from European house dust mites
TNACSINGNAPAEIDLRQMRTVTPIRMQGGCGSCWAFSGVAATESAYLAYRQQSLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 2 - protein, 222 aa, Artificial sequence
  2AS8 [SEQ ID NO 1], after one round of de-Antigenization
TNACSISGSAPAAIDLRQMRTVTPIRMQGGCGSCWAFSGVAATESAYLAYRQQSLDLAEQELVDCASTHGCHGDTIPAGIEYIQAAGVVQESYYAYVATAQSCAAPGAQRFGISNYCQIYPPNAAKIREALAQTHSAIAVIIGIKDLAAFAAYSGATIIQADSGYQPNYHAVNIVGYSTAQGVTYWIVRNSWSTGWGDGGYGYFAAGIDLMMIEEYPYVVIL

SEQ ID NO: 3 - protein, 222 aa, Artificial sequence
  2AS8 [SEQ ID NO 1], after two rounds of de-Antigenization
TNACSISGSAPAAIDLRQMRTVTPIRMQGGCGSCWAFSGVAATESAYLAYRQQSLDLAEQELVDCASTHGCHGDTIPAGIEYIQAAGVVQESYYAYVATAQSCAAPGAQRFGISNYCQIYPPSAAKIREALAATHSAIAVIIGIKDLAAFAAYSGATIIQADSGYQPNYHAVNIVGYSTAQGVTYWIVRNSWSTGWGDGGYGYFAAGIDLMMIEEYPYVVIL

SEQ ID NO: 4 - protein, 346 aa, Juniperus ashei
  1PXZ, major pollen allergen, Jun a 1, from cedar
DNPIDSCWRGDSNWDQNRMKLADCAVGFGSSTMGGKGGDFYTVTSTDDNPVNPTPGTLRYGATREKALWIIFSQNMNIKLKMPLYVAGHKTIDGRGADVHLGNGGPCLFMRKVSHVILHSLHIHGCNTSVLGDVLVSESIGVEPVHAQDGDAITMRNVTNAWIDHNSLSDCSDGLIDVTLGSTGITISNNHFFNHHKVMLLGHDDTYDDDKSMKVTVAFNQFGPNAGQRMPRARYGLVHVANNNYDPWNIYAIGGSSNPTILSEGNSFTAPSESYKKEVTKRIGCESPSACANWVWRSTRDAFINGAYFVSSGKTEETNIYNSNEAFKVENGNAAPQLTKNAGVVT

SEQ ID NO: 5 - protein, 346 aa, Artificial sequence
  1PXZ [SEQ ID NO 4], after one round of de-Antigenization
SNPIDSCWRGDSNWDQNRMKLADCAVGFGSSTMGGKGGDFYTVTSTSDNPVNPTPGTLRYGATREKALWIIFSQNMTIKLKMPLYVAGHKTIDGRGASVHLGNGGPCLFMRKVSHVILHSLHIHGCSTSVLGDVLVSESIGVEPVHAQDGDAITMRNVTNAWIDHNSLSSCSDGLIDVTLGSTGITISNNHFFGHTKVMLLGHDDTYDDDKSMKVTVAFNQFGPGAGQRMPRARYGLVHVANNNYDPWGIYAIGGSSNPTILSEGNSFTAPSESYKKEVTKRIGCESPSACANWVWRSTADAFIGGAYFVSSGKTEETSIYNSGEAFKVANGAAAPQLTKGAGVVT

SEQ ID NO: 6 - protein, 346 aa, Artificial sequence
  1PXZ [SEQ ID NO 4], after two rounds of de-Antigenization
SNPIDSCWRGGSSWGTSRMKLAACAVGFGSSTMGGKGGTFYTVTSTSDNPVNPTPGTLRYGATREKALWIIFSQNMTIKLKMPLYVAGHKTIDGRGASVHLGNGGPCLFMRKVSHVILHSLHIHGCSTSVLGDVLVSESIGVEPVHAQDGDAITMRNVTNAWIDHNSLSSCSDGLIDVTLGSTGITISNNHFFGHTKVMLLGHDGTYAADKSMKVTVAFNQFGPGAGQRMPRARYGLVHVANNNYDPWGIYAIGGSSSPTILSEGNSFTAPSESYKKEVTKRIGCTSPSACAGWVWRSTADAFIGGAYFVSSGKTEETSIYNSGEAFTVANGAAAPQLTKGAGVVT

SEQ ID NO: 7 - protein, 346 aa, Artificial sequence
  1PXZ [SEQ ID NO 4], after three rounds of de-Antigenization
SNPIDSCWRGGSSWGTSRMKLAACAVGFGSSTMGGKGGTFYTVTSTSDSPVGPTPGTLRYGATRTKALWIIFSQSMTITLTMPLYVAGTKTIDGRGASVHLGNGGPCLFMRTVSHVILHSLHIHGCSTSVLGDVLVSESIGVEPVHAQDGDAITMRNVTNAWIDHNSLSSCSDGLIDVTLGSTGITISNNHFFGHTKVMLLGHDGTYAADASMKVTVAFNQFGPGAGQAMPRATYGLVHVANNNYDPWGIYAIGGSSSPTILSEGNSFTAPSESYKKEVTKRIGCTSPSACAGWVWRSTADAFIGGAYFVSSGKTEETSIYNSGEAFTVANGAAAPQLTKGAGVVT

SEQ ID NO: 8 - protein, 346 aa, Artificial sequence
  1PXZ [SEQ ID NO 4], after four rounds of de-Antigenization
SNPIDSCWRGGSSWGTSRMKLAACAVGFGSSTMGGKGGTFYTVTSTSDSPVGPTPGTLRYGATRTKALWIIFSQSMTITLTMPLYVAGTKTIDGRGASVHLGNGGPCLFMRTVSHVILHSLHIHGCSTSVLGDVLVSESIGVEPVHAQDGDAITMRNVTNAWIDHNSLSSCSDGLIDVTLGSTGITISNNHFFGHTTVMLLGHDGTYAADASMTVTVAFNQFGPGAGQAMPRATYGLVHVANNNYDPWGIYAIGGSSSPTILSEGNSFTAPSASYKKEVTKRIGCTSPSACAGWVWRSTADAFIGGAYFVSSGKTAATSIYSSGEAFTVANGAAAPQLTKGAGVVT

SEQ ID NO: 9 - protein, 204 aa, Vespula vulgaris
  1QNX, major allergen, Ves v 5, of yellow jackets
NNYCKIKCLKGGVHTACKYGSLKPNCGNKVVVSYGLTKQEKQDILKEHNDFRQKIARGLETRGNPGPQPPAKNMKNLVWNDELAYVAQVWANQCQYGHDTCRDVAKYQVGQNVALTGSTAAKYDDPVKLVKMWEDEVKDYNPKKKFSGNDFLKTGHYTQMVWANTKEVGCGSIKYIQEKWHKHYLVCNYGPSGNFKNEELYQTK

SEQ ID NO: 10 - protein, 204 aa, Artificial sequence
  1QNX [SEQ ID NO 9], after one round of de-Antigenization
NNYCKIKCLKGGVHTACKYGSLKPNCGNKVVVSYGLTKQEKQDILKEHNAFRQKIAAGLETRGGPGPQPPAKNMKNLVWNDELAYVAQVWANQCQYGHDTCRDVAKYQVGQNVALTGSTAAKYDDPVKLVKMWEAEVKAYNPTKKFSGNSFLKTGHYTQMVWAGTKEVGCGSIKYIQEKWHKHYLVCNYGPSGNFTGEALYQTT

SEQ ID NO: 11 - protein, 204 aa, Artificial sequence
  1QNX [SEQ ID NO 9], after two rounds of de-Antigenization
GSYCAITCLKGGVHTACKYGSLTPSCGNKVVVSYGLTKQEKQDILKEHNAFRQKIAAGLETRGGPGPQPPAKNMKNLVWNDELAYVAQVWANQCQYGTDTCRDVAKYQVGQNVALTGSTAAKYDDPVKLVKMWEAEVKAYNPTKKFSGNSFLKTGHYTQMVWAGTKEVGCGSIKYIQEKWHKHYLVCNYGPSGNFTGEALYQTT

SEQ ID NO: 12 - protein, 204 aa, Artificial sequence
  1QNX [SEQ ID NO 9], after three rounds of de-Antigenization
GSYCAITCLKGGVHTACKYGSLTPSCGNKVVVSYGLTAAEKADILAEHNAFRAKIAAGLETRGGPGPQPPAKNMKNLVWNDELAYVAQVWANQCQYGTDTCRDVATYAVGQNVALTGSTAAKYDSPVALVAMWEAEVKAYNPTKKFSGNSFLKTGHYTQMVWAGTTEVGCGSIKYIQEKWHKHYLVCNYGPSGNFTGEALYQTT

SEQ ID NO: 13 - protein, 204 aa, Artificial sequence
  1QNX [SEQ ID NO 9], after four rounds of de-Antigenization
GSYCAITCLKGGVHTACKYGSLTPSCGNKVVVSYGLTAAEKAAILAEHNAFRAKIAAGLETRGGPGPQPPAKNMKNLVWNDELAYVAQVWANQCQYGTDTCRDVATYAVGQNVALTGSTAAKYSSPVALVAMWEAEVKAYNPTKKFSGNSFLKTGHYTQMVWAGTTEVGCGSIKYIQEKWHKHYLVCNYGPSGNFTGEALYQTT